(19) United States
(12) Patent Application Publication
(10) Pub. No.: US 2018/0282759 A1
(43) Pub. Date: Oct. 4, 2018

(54) SYSTEM FOR OVER-EXPRESSING TARGET PROTEIN AND METHOD FOR OVER-EXPRESSING TARGET PROTEIN

(71) Applicant: NATIONAL TSING HUA UNIVERSITY, HSINCHU (TW)

(72) Inventors: Yu-Chen HU, Hsinchu (TW); Chih-Che SHEN, Hsinchu (TW)

(21) Appl. No.: 15/686,167

(22) Filed: Aug. 25, 2017

(30) Foreign Application Priority Data
Mar. 31, 2017 (TW) 106111235

(51) Int. Cl. C12N 15/85; C12P 21/00; C12N 9/06; C12N 9/22; C12N 15/11

(52) U.S. Cl. CPC C12N 15/85; C12P 21/00; C12N 2310/20; C12N 9/22; C12N 15/11; C12N 9/003

(57) ABSTRACT

The present disclosure relates to a system for over-expressing a target protein. The system for over-expressing the target protein includes a dihydrofolate reductase (DHFR)-deficient CHO cell, an antifolate analog, a target protein expression plasmid and a CRISPRi expression plasmid. The target protein expression plasmid includes a target protein expression cassette and a DHFR expression cassette. The CRISPRi expression plasmid includes a gRNA cassette and a dCas9 expression cassette. The present disclosure also relates to a method for over-expressing the target protein. The method for over-expressing the target protein includes constructing the target protein expression plasmid, constructing the CRISPRi expression plasmid, establishing a first stable cell line, establishing a second stable cell line and performing a gene amplification.

### RELATED APPLICATIONS

This application claims priority to Taiwan Application Serial Number 106111235, filed Mar. 31, 2017, which is herein incorporated by reference.

### SEQUENCE LISTING

The sequence listing submitted via EFS, in compliance with 37 CFR § 1.52(e)(5), is incorporated herein by reference. The sequence listing text file submitted via EFS contains the file “CP-3635-US_Sequence Listing”, created on Jul. 25, 2017, which is 13,406 bytes in size.

### BACKGROUND

### Technical Field

The present disclosure relates to a DNA recombination technology. More particularly, the present disclosure relates to a DNA recombination technology which introduces foreign genetic materials using vectors.

### Description of Related Art

Many diseases are associated with the lack of certain proteins because various proteins in the body control the physiological state. Protein drugs are macromolecule drugs that can be defined as formulated proteins for the treatment of human diseases by in vitro administration. The raw materials for the protein drugs are mainly based on natural biological materials including the human body, animals, plants and microorganisms. Because of advantages of low toxicity and compatibility with the human body, protein drug becomes the current development trend of new drugs and one of the important projects for the development biological agent industry.

The main sources of the protein drugs in the past were extracted from human (blood or urine) or animal organs (such as pancreas). The yield of this method is very low and the source is not easy to obtain, so that the cost of this method is very high. Furthermore, a variety of infectious diseases such as AIDS and mad cow disease are prevalent, it is difficult to ensure that these protein drugs obtained from this method are not polluted by pathogens.

Genetically engineered drugs are manufactured by using biological cells, which can be screened in the laboratory to ensure that they are not contaminated with pathogens. In addition, the strong promoter can be used to enhance transgenic protein gene expression, thereby increasing protein production. At the outset of the genetic engineering, Escherichia coli and yeast are often used as host cells. These cells are easier to cultivate and enlarge the scale of production by biochemical reactors and their media are cheaper, hence their productions are large. However, Escherichia coli and yeast are lower living being organisms, some proteins produced by Escherichia coli or yeast can not be properly folded into the correct three-dimensional shape or can not undergo appropriate post-translational modification. Accordingly, these proteins lack their functions, or the shapes, functions, stabilities and immune properties of these proteins are affected. Therefore, the proteins produced by bacteria may not be able to achieve the required efficacy.

In this situation, mammal cells such as Chinese hamster ovary (CHO) cells, human embryonic kidney (HEK) cells, and African green monkey kidney (Vero) cells can be used as production tools to express proteins required more precise modification. The CHO cell is immortal and can be subcultured more than 100 generations. The type of glycosylation of the CHO cell is same as that of human cell. In addition, the CHO cell is very favorable for target protein separation and purification because it is a fibroblast, a non-secretory cell, and rarely secrets CHO endogenous protein. Therefore, the CHO cell is an ideal host for expressing complex biological macromolecules. At present, CHO cell gene amplification system is often used for the production of the target protein. In previous studies, the gradual increase in drug screening pressure can increase the copy number of the target gene during using the CHO cell gene amplification system for gene amplification. But gradually increasing the concentration of drugs is time-consuming and laborious, and it often takes more than a few months to screen out high-yield cell lines. Therefore, how to effectively improve the target protein production and shorten the screening time of high yield cell lines is a very important issue.

### SUMMARY

According to one aspect of the present disclosure, a system for over-expressing a target protein is provided. The system for over-expressing the target protein includes a dihydrofolate reductase (DHFR)-deficient CHO cell, an antifolate analog, a target protein expression plasmid and a CRISPRi expression plasmid. The target protein expression plasmid includes a target protein expression cassette and a DHFR expression cassette, wherein the target protein expression cassette includes a first promoter and a target protein gene, and the DHFR expression cassette includes a second promoter and a DHFR gene. The CRISPRi expression plasmid includes a gRNA cassette and a dCas9 expression cassette, wherein the gRNA cassette includes a third promoter, a gRNA sequence and a terminator, and the dCas9 expression cassette includes a fourth promoter, a dCas9-KRAB gene and an antibiotic resistance gene.

According to another aspect of the present disclosure, a method for over-expressing a target protein includes steps as follows. A target protein expression plasmid is constructed. The target protein expression plasmid includes a target protein expression cassette and a DHFR expression cassette, wherein the target protein expression cassette includes a first promoter and a target protein gene, and the DHFR expression cassette includes a second promoter and a DHFR gene. A CRISPRi expression plasmid is constructed. The CRISPRi expression plasmid includes a gRNA cassette and a dCas9 expression cassette, wherein the gRNA cassette includes a third promoter, a gRNA sequence and a terminator, and the dCas9 expression cassette includes a fourth promoter, a dCas9-KRAB gene and an antibiotic resistance gene. A first stable cell line is established by transfecting the target protein expression plasmid into a DHFR-deficient CHO cell and then screening with a screen medium to obtain the first stable cell line. A second stable cell line is established by transfecting the CRISPRi expression plasmid into the first stable cell line and then screening with an antibiotic to obtain the second stable cell line. A gene amplification is performed by culturing the second stable cell line in a medium containing an antifolate analog for over-expressing the target protein.

### BRIEF DESCRIPTION OF THE DRAWINGS

The present disclosure can be more fully understood by reading the following detailed description of the embodiment, with reference made to the accompanying drawings as follows:

FIG. 1A is a schematic view showing a construction of a pDHFR-2A-EGFP plasmid;

FIG. 1B is a schematic view showing a construction of a CRISPRi expression plasmid;

FIG. 2 is analytical results showing the effect of the CRISPRi expression plasmid and a green fluorescence test model on dhfr mRNA expression of CHO cells;

FIGS. 3A and 3B are analytical results showing the effect of the CRISPRi expression plasmid and the green fluorescence test model on green fluorescence expression of the CHO cells;

FIG. 4 is a schematic view showing a construction of a target protein expression plasmid;

FIG. 5 is analytical results showing dCas9 mRNA expression of second stable cell line;

FIGS. 6A and 6B are analytical results showing effect on growth rate of the second stable cell line;

FIG. 7 is a flow diagram showing a method for over-expressing a target protein according to another embodiment of the present disclosure;

FIGS. 8A to 8D are analytical results showing that the method for over-expressing the target protein of the present disclosure can increase target protein production;

FIGS. 9A to 9D are analytical results showing that the method for over-expressing the target protein of the present disclosure can enhance target protein gene expression; and

FIGS. 10A to 10D are analytical results showing that the method for over-expressing the target protein of the present disclosure can augment target protein gene amplification.

### DETAILED DESCRIPTION

The term “CRISPRi” refers to CRISPR interference system, which is a modified type II CRISPR/Cas9 system derived from the Streptococcus pyogenes. The Cas9 protein is modified to lose its endonuclease activity (RuvC1 and HNH), known as dCas9 (Cas9 D10A and H841A). The action principle of the CRISPRi system is the same as the type II CRISPR/Cas9 system, wherein the dCas9 protein binds to the target sequence of the target gene by an induction of the sgRNA or crRNA-trancrRNA complex, but the dCas9 protein does not cleave the target gene. Therefore, it can be used to block the RNA polymerase performing a gene transcription and inhibit an expression of the target gene.

Reference will now be made in detail to the present embodiments of the present disclosure, examples of which are illustrated in the accompanying drawings. Wherever possible, the same reference numbers are used in the drawings and the description to refer to the same or like parts.

### Examples

### I. The System for Over-Expressing the Target Protein of the Present Disclosure

The system for over-expressing the target protein includes a DHFR-deficient CHO cell, an antifolate analog, a target protein expression plasmid and a CRISPRi expression plasmid.

The DHFR-deficient CHO cell can be a DUXB11 cell line or a DG44 cell line.

The antifolate analog can be Methotrexate (MTX) or Methionine sulfoximine (MSX).

The target protein expression plasmid includes a target protein expression cassette and a DHFR expression cassette, wherein the target protein expression cassette includes a first promoter and a target protein gene, and the DHFR expression cassette includes a second promoter and a DHFR gene. The first promoter can be CMV promoter or SV40 promoter. The second promoter can be CMV promoter or SV40 promoter, and the second promoter and the first promoter are different.

The CRISPRi expression plasmid includes a gRNA cassette and a dCas9 expression cassette, wherein the gRNA cassette includes a third promoter, a gRNA sequence and a terminator, and the dCas9 expression cassette includes a fourth promoter, a dCas9-KRAB gene and an antibiotic resistance gene. The dCas9 expression cassette can further include a 2A peptide sequence for linking the dCas9-KRAB gene and the antibiotic resistance gene. The third promoter can be U6 promoter, the fourth promoter can be CMV promoter or SV40 promoter, and the antibiotic resistance gene can be Zeocin resistance (Zeo^{R}) gene.

### 1.1 Construction of the CRISPRi Expression Plasmid and Establishment of Green FIuorescence Test Model

The effectiveness of CRISPRi for repressing DHFR in the CHO cells is not reported in previous studies. Therefore, this example first evaluates whether the CRISPRi can be used to effectively repress the expression of the DHFR gene in the CHO cells. A green fluorescent protein (egfp) gene is used as a reporter gene to construct a pDHFR-2A-EGFP plasmid co-expressing DHFR and green fluorescent protein. The CRISPRi expression plasmid of the system for over-expressing the target protein of the present disclosure is also constructed to establish the CRISPRi expression plasmid and the green fluorescence test model.

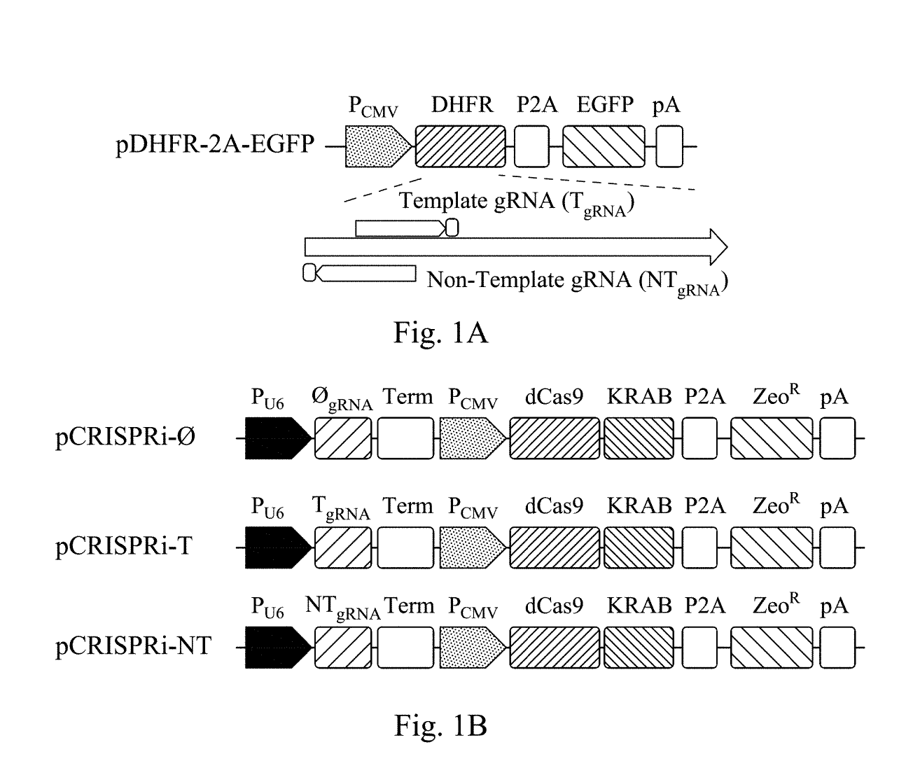

FIG. 1A is a schematic view showing a construction of the pDHFR-2A-EGFP plasmid. The pDHFR-2A-EGFP plasmid harbors an expression cassette consisting of CMV promoter, DHFR and egfp genes which are linked by a self-cleavage sequence (P2A peptide), so that EGFP could be co-translated with DHFR and served as a reporter for evaluating. The nucleotide sequence of the CMV promoter is referenced as SEQ ID NO: 1, the nucleotide sequence of the DHFR gene is referenced as SEQ ID NO: 2, the nucleotide sequence of the egfp gene is referenced as SEQ ID NO: 3, and the nucleotide sequence of the P2A peptide is referenced as SEQ ID NO: 4.

FIG. 1B is a schematic view showing a construction of the CRISPRi expression plasmid. According to one example of this embodiment; the CRISPRi expression plasmid is constructed using pUseAmp(+) (Merck Millipore) as a backbone and inserting into the gRNA cassette and the dCas9 expression cassette. The gRNA cassette includes the U6 promoter, the gRNA sequence and the terminator, wherein the gRNA cassette is initiated gRNA transcription by the U6 promoter. The nucleotide sequence of the U6 promoter is referenced as SEQ ID NO: 5, and the nucleotide sequence of the terminator is referenced as SEQ ID NO: 6. The dCas9 expression cassette includes the CMV promoter, the dCas9-KRAB gene, the Zeocin resistance (Zeo^{R}) gene and BGH polyA, wherein the dCas9 expression cassette is initiated dCas9-KRAB transcription by the CMV promoter, and the dCas9-KRAB gene and the Zeo^{R} gene are linked by the P2A peptide. The KRAB (Krüppel associated box) is a transcription repression domain that augments the dCas9 inhibition. The nucleotide sequence of the CMV promoter is referenced as SEQ ID NO: 1, and the nucleotide sequence of the dCas9-KRAB gene is referenced as SEQ ID NO: 7, wherein the sequence of dCas9-KRAB gene includes the sequence of dCas9 gene, SV40 nuclear localization sequence (tags dCas9 protein expressing in cytoplasm for importing into the cell nucleus), 3×HA flag sequence (for labeling in subsequent tests) and the sequence of KRAB gene. The nucleotide sequence of the Zeo^{R} gene is referenced as SEQ ID NO: 8, and the nucleotide sequence of the BGH polyA is referenced as SEQ ID NO: 9. A series of the CRISPRi expression plasmids, a pCRISPRi-Ø plasmid, a pCRISPRi-T plasmid, and a pCRISPRi-NT plasmid, are constructed in this example, wherein the pCRISPRi-Ø plasmid expresses scramble gRNA as a Ø group, the pCRISPRi-T plasmid expresses gRNA suppressed the template strand of DHFR gene as a T group and the pCRISPRi-NT plasmid expresses gRNA targeted the non-template strand of DHFR gene as a NT group. These CRISPRi expression plasmids differ in the sequence of the gRNA and other part of these CRISPRi expression plasmids are the same. The nucleotide sequence of the gRNA of the Ø group is referenced as SEQ ID NO: 10, the nucleotide sequence of the gRNA of the T group is referenced as SEQ ID NO: 11, and the nucleotide sequence of the gRNA of the NT group is referenced as SEQ ID NO: 12.

The pDHFR-2A-EGFP plasmid and one of the CRISPRi expression plasmid are co-transfected into CHO DUXB11 cell line (commercially obtained from the bioresource collection and research center, BCRC). To calculate the efficiency of CRSIPRi for suppressing DHFR expression, the change of dhfr mRNA expression in the transfected cells is analyzed by qRT-PCR and the expression of the green fluorescent protein is analyzed by fluorescence microscopy and flow cytometry at 48 hours post-transfection. The nucleotide sequence of the forward primer (Q mDHFR F) and the reverse primer (Q mDHFR R) used in qRT-PCR is referenced as SEQ ID NO: 13 and SEQ ID NO: 14 respectively.

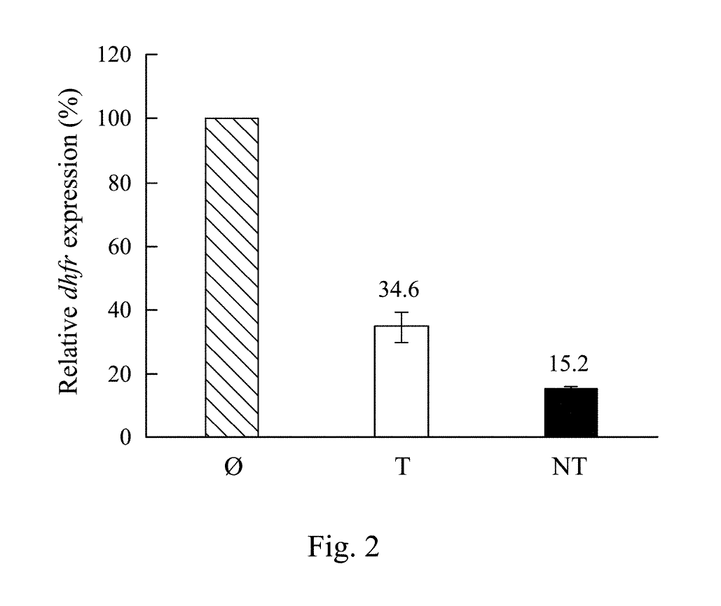

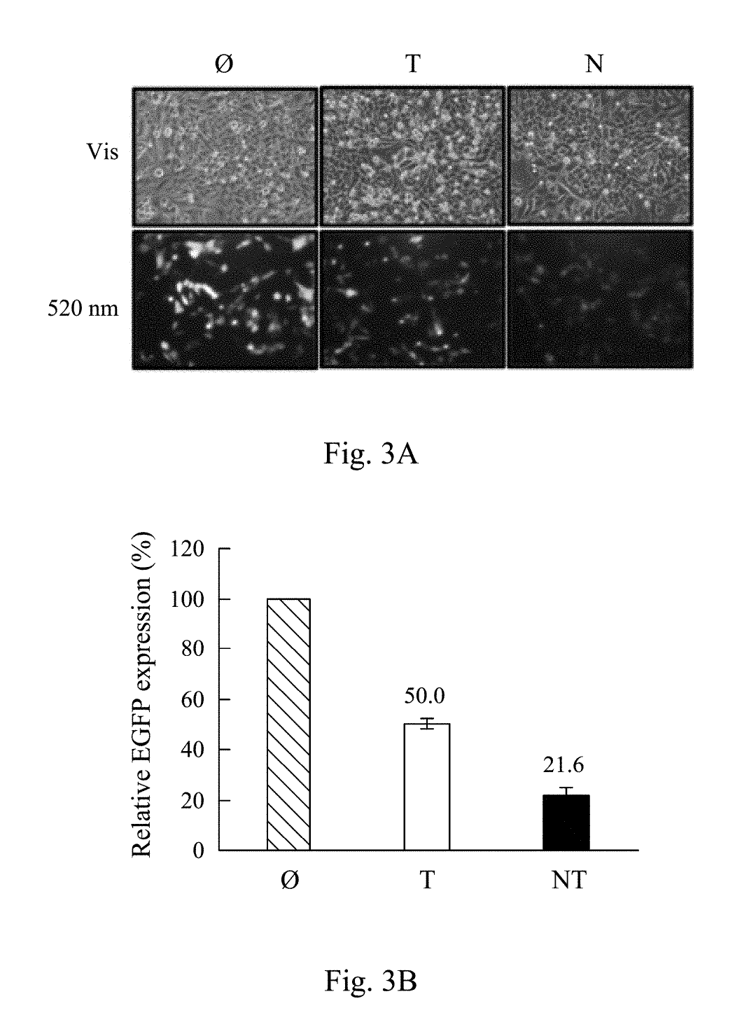

FIG. 2 is analytical results showing the effect of the CRISPRi expression plasmid and the green fluorescence test model on dhfr mRNA expression of the CHO cells, wherein the dhfr mRNA expression of the Ø group is used as a baseline to calculate a relative dhfr mRNA expression of the T group and the NT group. FIGS. 3A and 3B are analytical results showing the effect of the CRISPRi expression plasmid and the green fluorescence test model on green fluorescence expression of the CHO cells, wherein FIG. 3A shows optical and fluorescence microscopic images, and FIG. 3B shows fluorescence intensity analytical results of the flow cytometry. In FIG. 3B, the fluorescence intensity of the Ø group is used as a baseline to calculate a relative fluorescence intensity of the T group and the NT group.

In FIG. 2, the CHO cells transfected with pCRISPRi-T plasmid (T group) and pCRISPRi-NT plasmid (NT group) expressed only 34.6%±4.3% and 15.2%±0.4% of dhfr, when compared with the cells transfected with pCRISPRi-Ø plasmid (Ø group). The DHFR suppression rate of the T group and the NT group is 66%±4.3% and 85%±0.4% respectively, which has significant difference (p<0.05). In FIG. 3A, the EGFP in the T group and the NT group is significantly lower than that in the Ø group. In FIG. 3B, the T and NT groups express only 50%±1.6% and 21.6%±2.8% of the EGFP relative to the 0 group. The DHFR suppression rate of the T group and the NT group is 50%±1.6% and 79%±2.8% respectively, which has significant difference (p<0.05).

These data confirm that the CRISPRi expression plasmid established by the present disclosure can effectively suppress the expression of DHFR gene in the CHO cells. The gene transcription suppression efficiency is up to 85%±0.4%, and the protein suppression efficiency is up to 79%. The efficiency of RNAi for suppressing the DHFR expression in the conventional manner is about 72%. In contrast, the CRISPRi system of the present disclosure has a higher inhibitory efficiency.

### 1.2 Establishment of the System for Over-Expressing the Target Protein

It is confirmed from Example 1.1 that the CIRSPRi expression plasmid of the present disclosure effectively suppresses the expression of the DHFR gene in the CHO cells. It is expected that the CRISPRi-mediated dhfr suppression could further enhance the gene amplification. In this example, the target protein expression plasmid of the present disclosure is further constructed to establish the system for over-expressing the target protein which can enhance the target protein production by the gene amplification.

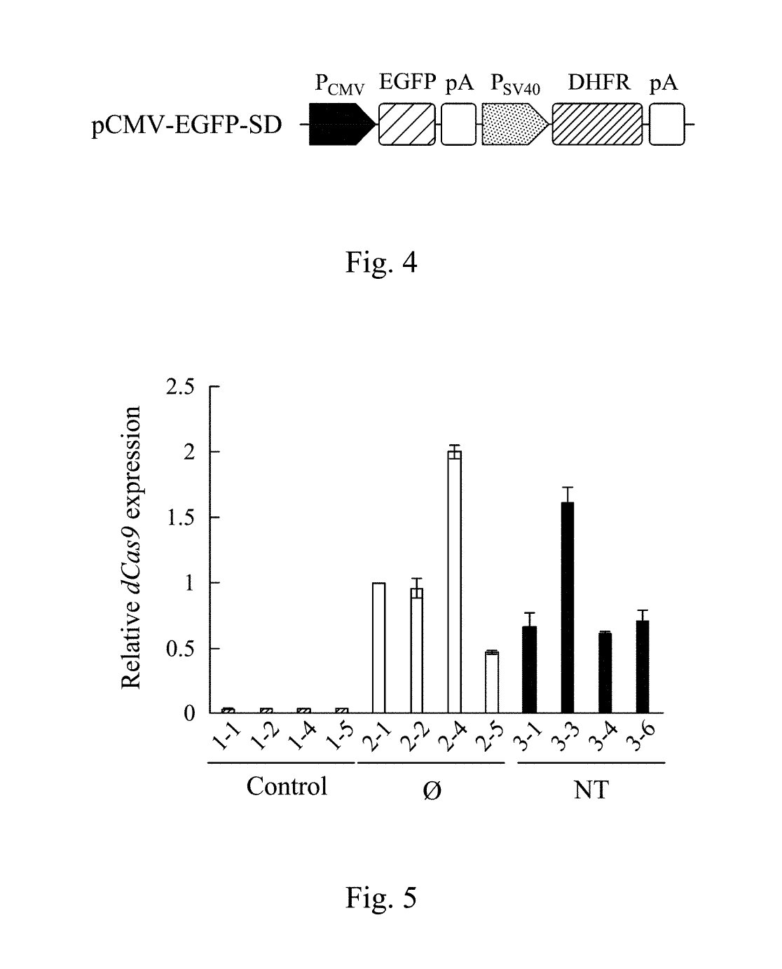

FIG. 4 is the schematic view showing the construction of the target protein expression plasmid. According to one example of this embodiment, the target protein expression plasmid is a pCMV-EGFP-SD plasmid, and the targert protein is the EGFP as a test model. It is to be noted that the EGFP is one embodiment of the present disclosure, and the target protein gene can be changed depending on the target protein desired to be produced. The pCMV-EGFP-SD plasmid is constructed using pUseAmp(+) (Merck Millipore) as the backbone and inserting the target protein expression cassette and the DHFR expression cassette. The target protein expression cassette includes the CMV promoter, the egfp gene and the BGH polyA sequence. The DHFR expression cassette includes the SV40 promoter, the DHFR gene and the BGH polyA. The target protein expression cassette and the DHFR expression cassette are initiated transcription by the CMV promoter and the SV40 promoter, respectively. The nucleotide sequence of the CMV promoter is referenced as SEQ ID NO: 1, the nucleotide sequence of the egfp gene is referenced as SEQ ID NO: 3, the nucleotide sequence of the BGH polyA is referenced as SEQ ID NO: 9, the nucleotide sequence of the DHFR gene is referenced as SEQ ID NO: 2, and the nucleotide sequence of the SV40 promoter is referenced as SEQ ID NO: 15.

The CHO DUXB11 cells are transfected with the pCMV-EGFP-SD plasmid and cultured using nucleoside-free α-MEM to select EGFP-expressing stable clones. Then the first stable cell line expressing EGFP is selected by the fluorescence microscope. The first stable cell line is transfected with the CRSIRPi expression plasmid and cultured using Zeocin to select the second stable cell line with co-integrated DHFR and EGFP genes. There are three groups in this example. For mimicking the conventional method, the first stable cell line is cultured in parallel without transfecting the CRSIRPi expression plasmid as the control group. For comparing the effect of the dCas9 protein and gRNA expression on the target protein production, the first stable cell line is transfected with the pCRISPRi-Ø plasmid as the Ø group. For confirming whether CRISPRi-mediated specific DHFR suppression can enhance the target protein production, the first stable cell line is transfected with the pCRISPRi-NT plasmid as the NT group. After screening the second stable cell lines of the control group, the Ø group and the NT group, the relative quantitative analysis of qRT-PCR is used to analyze whether these second stable cell lines express dCas9 mRNA. The nucleotide sequence of the forward primer (Q dCas9 F) and the reverse primer (Q dCas9 R) used in qRT-PCR is referenced as SEQ ID NO: 16 and SEQ ID NO: 17 respectively.

FIG. 5 is analytical results showing the dCas9 mRNA expression level of the second stable cell line, wherein the dCas9 mRNA expression of the second stable cell line 2-1 is used as the baseline to calculate the relative dCas9 mRNA expression of other second stable cell line. In FIG. 5, the second stable cell lines transfected with the CRISPRi expression plasmid (the Ø group and the NT group) stably express the dCas9 gene.

### 1.3 the Effect of the System for Over-Expressing the Target Protein of the Present Disclosure on Cell Growth Rate

To examine whether the CRISPRi-mediated DHFR suppression affects cell growth, the second stable cell lines of the control group, the Ø group and the NT group in Example 1.2 are seeded to 6-well plates (1×10⁵ cells/well). The cell number of attached cells is calculated every other day, and the cell numbers at the same time points for all 4 clones in the same group are averaged. The doubling time of the cells is calculated using the cell density of the logarithmic growth phase (48-120 hours).

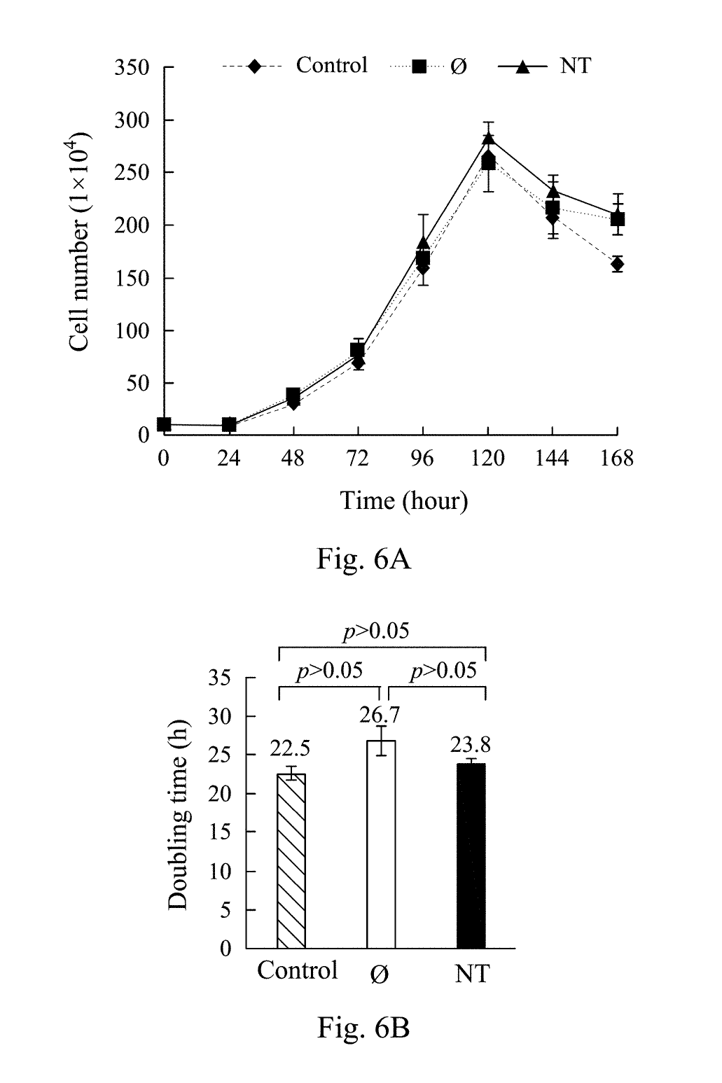

FIGS. 6A and 6B are analytical results showing effect on growth rate of the second stable cell line, wherein FIG. 6A is the growth curve of the second stable cell line, and FIG. 6B is the doubling time chart of the second stable cell line. In FIG. 6A, the second stable cell lines of all 3 groups have virtually overlapped growth curves. In FIG. 6B, the doubling time of the second stable cell line is 22.5±0.8 hours, 26.7±1.9 hours and 23.8±0.6 hours for the control group, the Ø group and the NT group, respectively. Although the doubling time of the second stable cell line of the NT group is longer than that of the control group, there is no significant difference between groups (p>0.05). These data indicate that the system for over-expressing the target protein of the present disclosure does not affect the growth rate of the CHO cells.

### II. A Method for Over-Expressing the Target Protein of the Present Disclosure

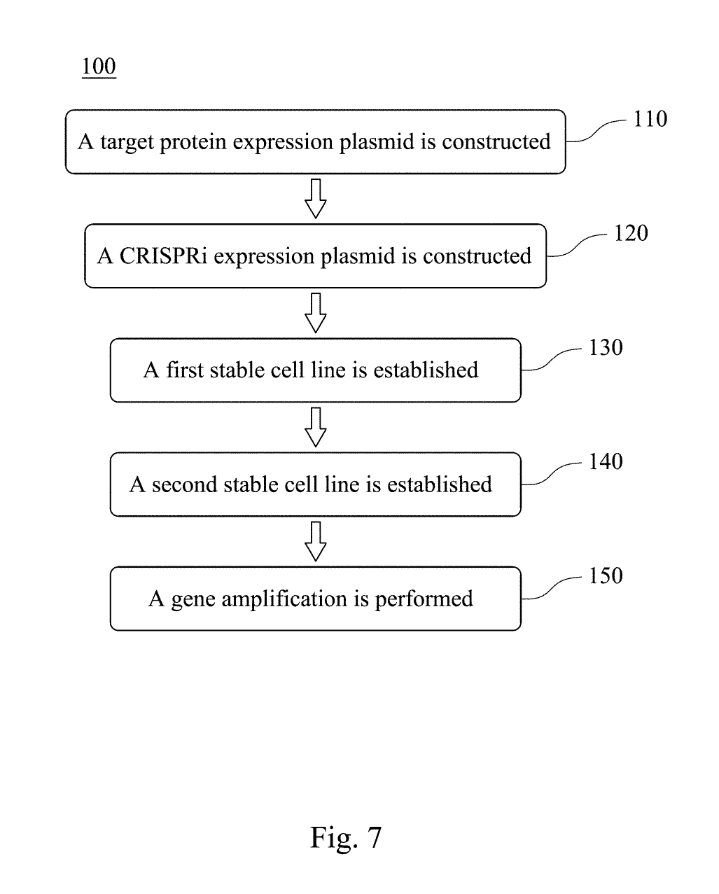

FIG. 7 is a flow diagram showing the method for over-expressing the target protein 100 according to another embodiment of the present disclosure. In FIG. 7, the method for over-expressing the target protein 100 includes a step 110, a step 120, a step 130, a step 140 and a step 150.

In the step 110, the target protein expression plasmid is constructed. The target protein expression plasmid includes the target protein expression cassette and the DHFR expression cassette, wherein the target protein expression cassette includes the first promoter and the target protein gene, and the DHFR expression cassette includes the second promoter and the DHFR gene. The first promoter can be CMV promoter or SV40 promoter. The second promoter can be CMV promoter or SV40 promoter, and the second promoter and the first promoter are different.

In the step 120, the CRISPRi expression plasmid is constructed. The CRISPRi expression plasmid includes the gRNA cassette and the dCas9 expression cassette, wherein the gRNA cassette includes the third promoter, the gRNA sequence and the terminator, and the dCas9 expression cassette includes the fourth promoter, the dCas9-KRAB gene and the antibiotic resistance gene. The dCas9 expression cassette can further include the 2A peptide sequence for linking the dCas9-KRAB gene and the antibiotic resistance gene. The third promoter can be U6 promoter, the fourth promoter can be CMV promoter or SV40 promoter, and the antibiotic resistance gene can be Zeocin resistance (Zeo^{R}) gene.

In the step 130, the first stable cell line is established by transfecting the target protein expression plasmid into the DHFR-deficient CHO cell and then screening with a screen medium to obtain the first stable cell line. The DHFR-deficient CHO cell can be the DUXB11 cell line or the DG44 cell line. Transfection can be done using calcium phosphate transfection, electroporation or liposome transfection. The screen medium can be a nucleoside-free α-MEM.

In the step 140, the second stable cell line is established by transfecting the CRISPRi expression plasmid into the first stable cell line and then screening with an antibiotic to obtain the second stable cell line. The antibiotic can be Zeocin.

In the step 150, a gene amplification is performed by culturing the second stable cell line in a medium containing the antifolate analog for over-expressing the target protein. The antifolate analog can be Methotrexate (MTX) or Methionine sulfoximine (MSX).

### 2.1 the Method for Over-Expressing the Target Protein of the Present Disclosure Increases Target Protein Production

This example further evaluates whether the method for over-expressing the target protein of the present disclosure can increase the target protein production. The CHO DUXB11 cells are transfected with the pCMV-EGFP-SD plasmid and cultured using nucleoside-free α-MEM to select the first stable cell line. The first stable cell line is transfected with the CRSIRPi expression plasmid and cultured using Zeocin to select the second stable cell line with co-integrated DHFR and EGFP genes. Then the second stable cell line is performed the gene amplification by culturing in the medium containing MTX. There are three groups in this example. The first stable cell line is cultured in parallel without transfecting the CRSIRPi expression plasmid as the control group. The first stable cell line is transfected with the pCRISPRi-Ø plasmid as the Ø group. The first stable cell line is transfected with the pCRISPRi-NT plasmid as the NT group. Each group selects 6 second stable cell lines to start the gene amplification, and using the gradual increase in the MTX concentration to achieve the effect of gene amplification. The MTX concentration is 50 nM at the beginning of the selection process. After 4 weeks of selection, the MTX concentration is raised to 250 nM and the selection process is repeated for another 4 weeks.

After completion of the gene amplification, each group selects 4 second stable cell lines using fluorescence microscopy and flow cytometry to analyze whether the EGFP successfully amplified in these second stable cell lines. The 4 second stable cell lines in the control group are second stable cell lines 1-1, 1-2, 1-4 and 1-5. The 4 second stable cell lines in the Ø group are second stable cell lines 2-1, 2-2, 2-4 and 2-5. The 4 second stable cell lines in the NT group are second stable cell lines 3-1, 3-3, 3-4 and 3-6.

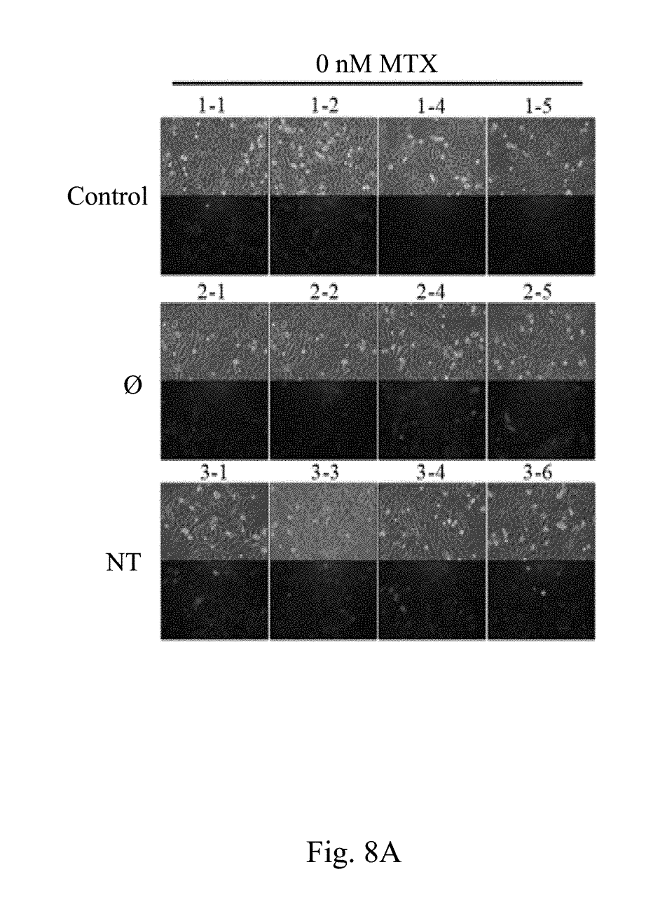

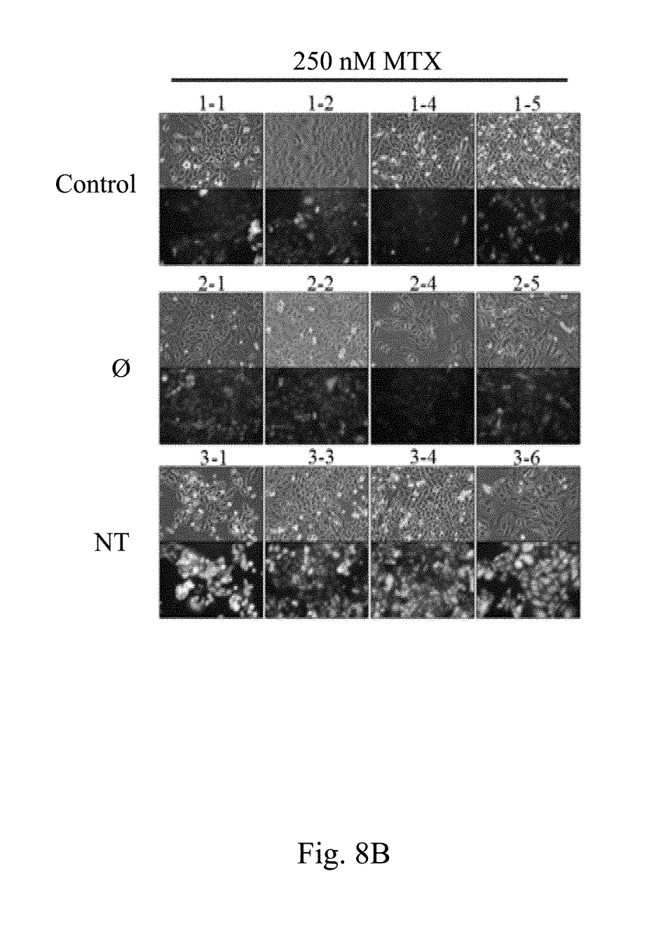

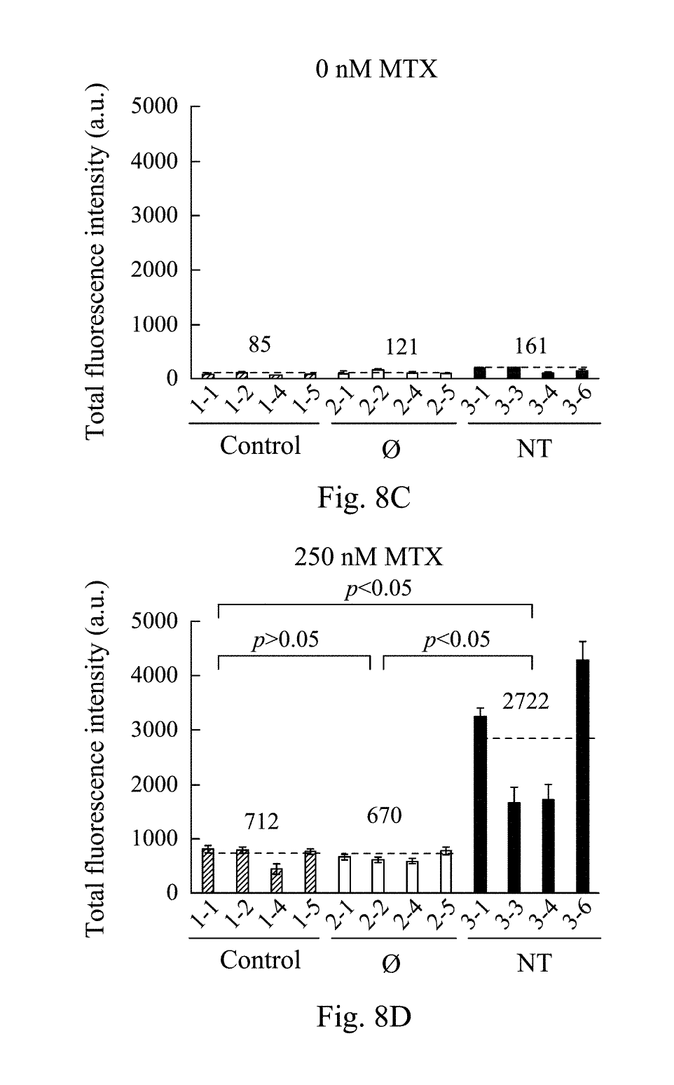

FIGS. 8A to 8D are analytical results showing that the method for over-expressing the target protein of the present disclosure can increase target protein production. FIG. 8A is a set of optical and fluorescence microscopic images showing the EGFP expression of the CHO cells before the gene amplification. FIG. 8B is a set of optical and fluorescence microscopic images showing the EGFP expression of the CHO cells after the gene amplification. FIG. 8C is a chart showing the EGFP total fluorescence intensity (FI) of the CHO cells before the gene amplification. FIG. 8D is a chart showing the EGFP total FI of the CHO cells after the gene amplification.

In FIGS. 8A and 8B, no remarkable differences in EGFP expression existed between second stable cell lines and between groups before the gene amplification (at 0 nM MTX), yet all 3 groups express apparently more EGFP after the gene amplification (at 250 nM MTX). Notably, the EGFP expression appears similar in the control group and the Ø groups but is much stronger in the NT group.

In FIGS. 8C and 8D, the total FI of each second stable cell line is further measured by flow cytometry and average values for each group are calculated. Before the gene amplification, the average total FI for the control group, the 0 group and the NT group is 85±9 a.u., 121±14 a.u. and 161±23 a.u. respectively, without significant difference (p>0.05) among three groups. After the gene amplification, the average total FI in the control group and the Ø group increase to 712±86 a.u. and 670±41 a.u., without significant difference (p>0.05) between these two groups, indicating that expression of dCas9 and scramble gRNA (0) does not enhance or mitigate the target protein production upon MTX selection. In contrast, the average total FI in the NT group remarkable rises to 2722±632 a.u. with significant difference (p<0.05).

These data attest that the DHFR suppression by the method for over-expressing the target protein of the present disclosure can increase the target protein production after the gene amplification. Besides, the EGFP production using the method for over-expressing the target protein of the present disclosure is increased up to 3.8-fold than that produced by the traditional method.

### 2.2 the Method for Over-Expressing the Target Protein of the Present Disclosure Enhances Target Protein Gene Expression

To evaluate whether the method for over-expressing the target protein of the present disclosure can enhance the target protein gene expression, the relative quantification of the egfp mRNA expression and the dhfr mRNA expression are further analyzed by qRT-PCR in this example, wherein the mRNA expression level in second stable cell line 2-1 of the Ø group before the gene amplification is used as the baseline. The primers used in qRT-PCR are Q EGFP F, Q EGFP R, Q mDHFR F and Q mDHFR R. The nucleotide sequence of the Q EGFP F, the Q EGFP R, the Q mDHFR F and the Q mDHFR R is referenced as SEQ ID NO: 18, SEQ ID NO: 19, SEQ ID NO: 13 and SEQ ID NO: 14 respectively.

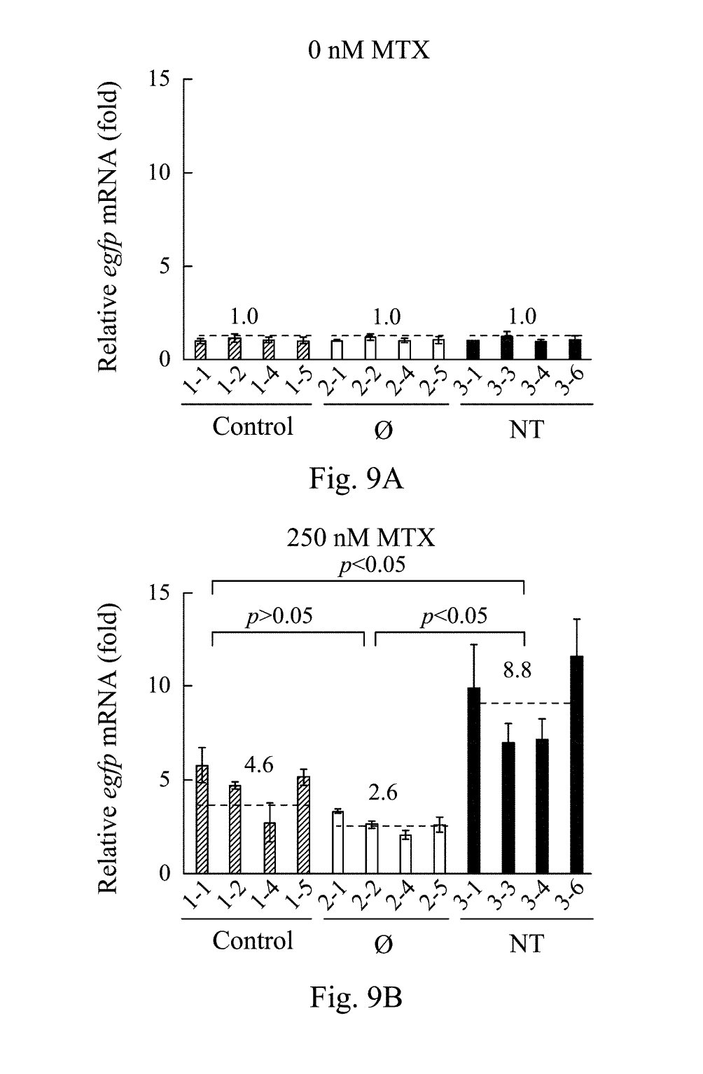

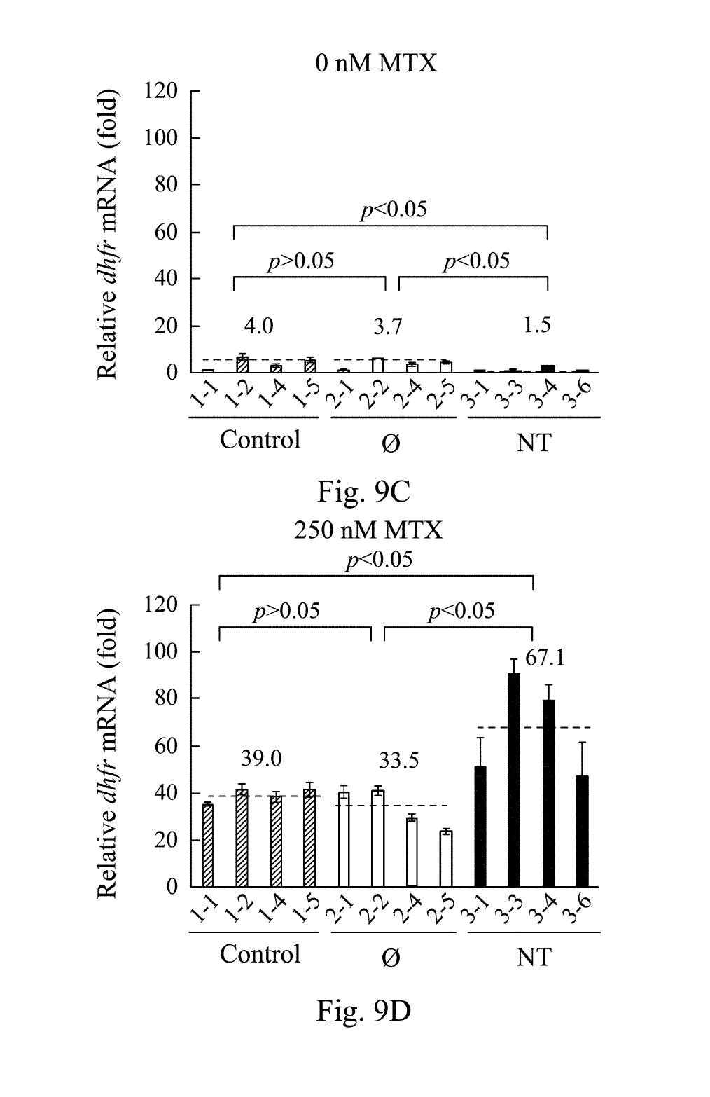

FIGS. 9A to 9D are analytical results showing that the method for over-expressing the target protein of the present disclosure can enhance target protein gene expression. FIG. 9A shows the relative egfp mRNA levels of the CHO cells before the gene amplification. FIG. 9B shows the relative egfp mRNA levels of the CHO cells after the gene amplification. FIG. 9C shows the relative dhfr mRNA levels of the CHO cells before the gene amplification. FIG. 9D shows the relative dhfr mRNA levels of the CHO cells after the gene amplification.

FIGS. 9A and 9B show the change degree of the egfp mRNA expression before and after the gene amplification. Before the gene amplification, the average relative egfp mRNA level for the control group, the Ø group and the NT group is 1.05±0.02 fold, 1.06±0.03 fold and 1.06±0.05 fold respectively, without significant difference (p>0.05) among three groups. After the gene amplification, the relative egfp mRNA levels increase to 4.6±0.2 fold and 2.6±0.1 fold in the control group and the Ø group, respectively, and further elevates to 8.8±0.3 fold in the NT group. The relative egfp mRNA level of the NT group is 1.94 fold higher than that of the control group, with statistically significant difference (p<0.05).

FIGS. 9C and 9D show the change degree of the dhfr mRNA expression before and after the gene amplification. Before the gene amplification, the average relative dhfr mRNA level for the control group, the Ø group and the NT group is 4.07±1.3 fold, 3.73±1.0 fold and 1.58±0.3 fold respectively, without significant difference (p>0.05) among three groups. After the gene amplification, the relative dhfr mRNA levels increase to 39.02±1.4 fold and 33.51±4.2 fold in the control group and the Ø group, respectively, and further elevates to 67.1±10.6 fold in the NT group. The relative dhfr mRNA level of the NT group is 1.7-fold higher than that of the control group, with statistically significant difference (p<0.05).

These data indicate that the DHFR suppression by the method for over-expressing the target protein of the present disclosure can increase the selective pressure and thereby increase the target protein production after the gene amplification. Under the expression of the pCRISPRi-NT plasmid, the EGFP gene expression is increased by 1.94 times and the DHFR gene expression is increased by 1.7 times, when compared with the traditional method (the control group).

### 2.3 the Method for Over-Expressing the Target Protein of the Present Disclosure Augments Target Protein Gene Amplification

In aforementioned examples, it is confirmed that the method for over-expressing the target protein of the present disclosure can effectively increase the target protein production and the gene expression level of the target protein. To examine whether the increased mRNA and protein levels arise from enhanced gene amplification, the absolute copy numbers of the EGFP gene and the DHFR gene per cell before and after the gene amplification are analyzed in this example.

The genomic DNA is extracted from the second stable cell lines using Genomic DNA mini kit (Geneaid). Q-PCR reactions are conducted with 6 ng genomic DNA and a primer set specific for the DHFR gene or the EGFP gene (Q mDHFR F, Q mDHFR R, Q EGFP F and Q EGFP R). To quantify the absolute gene copy number, the p-CMV-EGFP-2A-DHFR plasmid was serially diluted (4, 0.4, 0.04, 0.004, 0.0004 μg) and quantified by Q-PCR to generate the standard curve. The absolute DHFR and EGFP copy numbers per cell are then quantified based on the assumption that 6 ng total genomic DNA is equal to 1820 genomic DNA molecules.

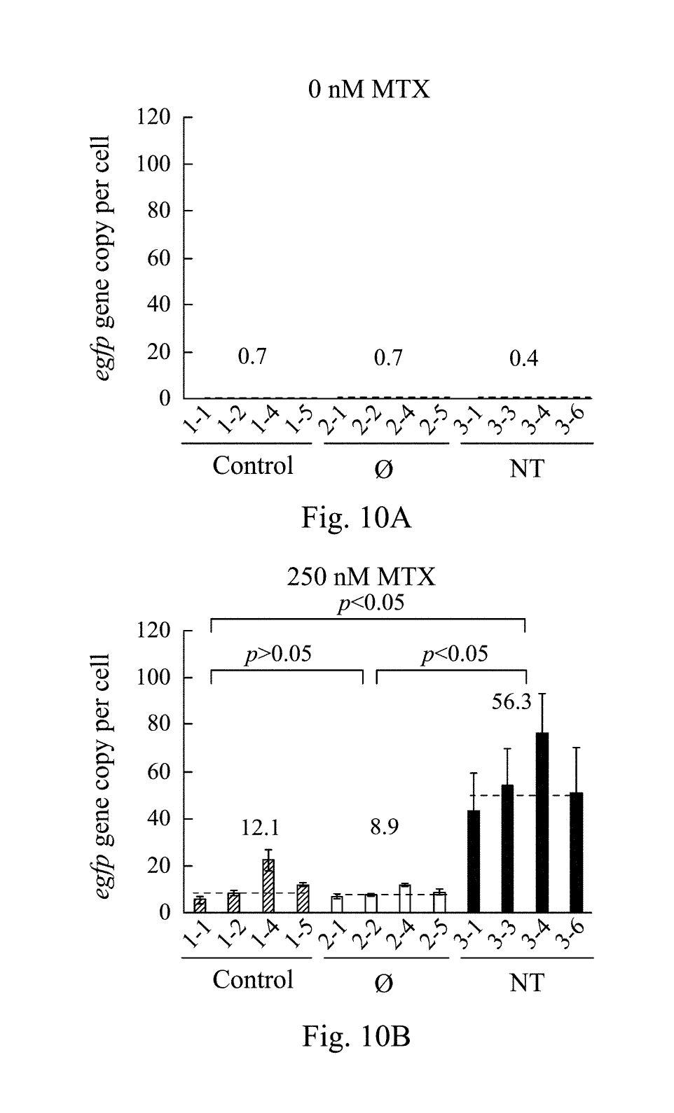

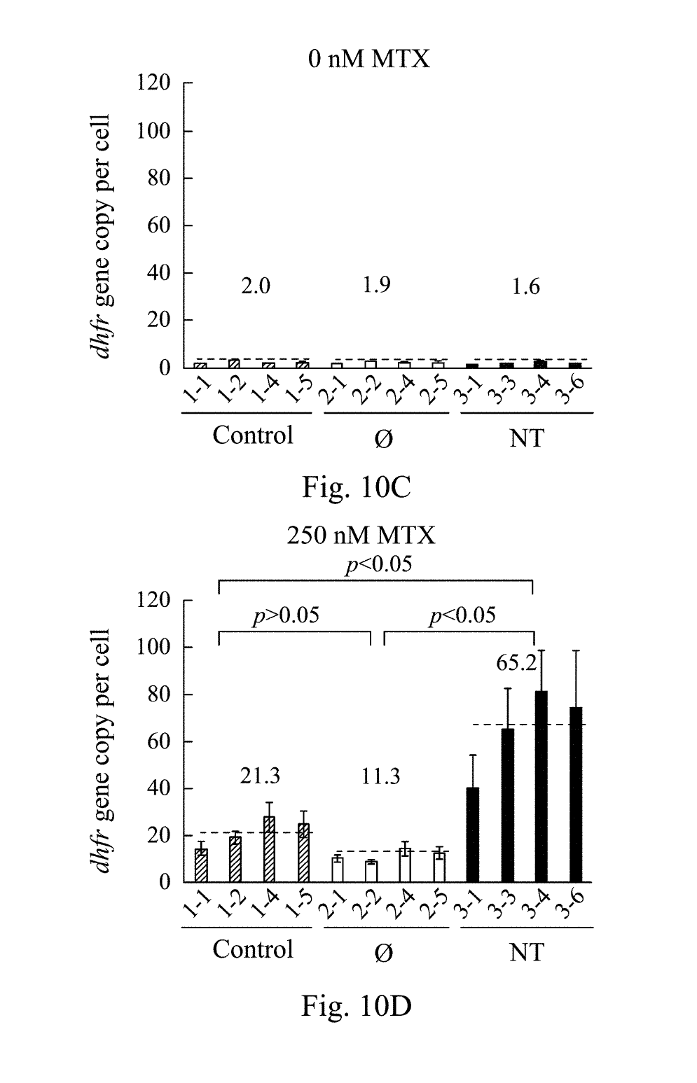

FIGS. 10A to 10D are analytical results showing that the method for over-expressing the target protein of the present disclosure can augment target protein gene amplification. FIG. 10A shows the gene copy number of EGFP per CHO cell before the gene amplification. FIG. 10B shows the gene copy number of EGFP per CHO cell after the gene amplification. FIG. 10C shows the gene copy number of DHFR per CHO cell before the gene amplification. FIG. 10D shows the gene copy number of DHFR per CHO cell after the gene amplification.

FIGS. 10A and 10B show the change of the EGFP gene copy number before and after the gene amplification. Before the gene amplification, the average EGFP gene copy number for the control group, the Ø group and the NT group is 0.73±0.04 per cell, 0.76±0.07 per cell and 0.46±0.05 per cell respectively, without significant difference (p>0.05) among three groups. After the gene amplification, the average EGFP gene copy number increase to 12.1±3.6 per cell and 8.9±1.07 per cell in the control group and the Ø group, respectively, and further elevates to 56.3±0.5 per cell in the NT group. The average EGFP gene copy number of the NT group is 3.5-fold higher than that of the control group, with statistically significant difference (p<0.05).

FIGS. 10C and 10D show the change of the DHFR gene copy number before and after the gene amplification. Before the gene amplification, the average DHFR gene copy number for the control group, the Ø group and the NT group is 2.0±0.3 per cell, 1.9±0.3 per cell and 1.6±0.2 per cell respectively, without significant difference (p>0.05) among three groups. After the gene amplification, the average DHFR gene copy number increase to 21.3±3.0 per cell and 11.3±1.5 per cell in the control group and the Ø group, respectively, and further elevates to 65.2±10.2 per cell in the NT group. The average DHFR gene copy number of the NT group is 3-fold higher than that of the control group, with statistically significant difference (p<0.05).

These data indicate that the DHFR suppression by the method for over-expressing the target protein of the present disclosure can improve the efficiency of the gene amplification. Under the expression of the pCRISPRi-NT plasmid, the EGFP gene amplification is increased by 3.5 times and the DHFR gene amplification is increased by 3 times, when compared with the traditional method (the control group).

Therefore, the system for over-expressing the target protein of the present disclosure and the method for over-expressing the target protein of the present disclosure can effectively suppress the DHFR gene expression in the CHO cells, wherein the suppression efficiency is 85%±0.4%. Accordingly, the CRISPRi-mediated suppression of DHFR gene can increase selective pressure and thereby increase the target protein production during the gene amplification. Compared with the traditional method, the system for over-expressing the target protein of the present disclosure and the method for over-expressing the target protein of the present disclosure can enhance the EGFP production for 3.8-fold, the egfp mRNA expression for 3.5-fold and the EGFP gene amplification for 3.5-fold. In addition, the system for over-expressing the target protein of the present disclosure and the method for over-expressing the target protein of the present disclosure do not affect the growth rate of the CHO cells. Furthermore, the gene amplification with 250 nM MTX selection in the system for over-expressing the target protein of the present disclosure and the method for over-expressing the target protein of the present disclosure can achieve same gene amplification effect of the traditional method using 1000 nM MTX selection, thereby shortening the time of the gene amplification. Therefore, the system for over-expressing the target protein of the present disclosure and the method for over-expressing the target protein of the present disclosure can significantly increase the target protein production and reduce the time of gene amplification.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the disclosure. In view of the foregoing, it is intended that the present disclosure cover modifications and variations of this disclosure provided they fall within the scope of the following claims.

What is claimed is:

1. A system for over-expressing a target protein, comprising:
a dihydrofolate reductase (DHFR)-deficient CHO cell;
an antifolate analog;
a target protein expression plasmid, comprising:
a target protein expression cassette, which comprises a first promoter and a target protein gene; and
a DHFR expression cassette, which comprises a second promoter and a DHFR gene; and
a CRISPRi expression plasmid, comprising:
a gRNA cassette, which comprises a third promoter, a gRNA sequence and a terminator; and
a dCas9 expression cassette, which comprises a fourth promoter, a dCas9-KRAB gene and an antibiotic resistance gene.

2. The system for over-expressing a target protein of claim 1, wherein the DHFR-deficient CHO cell is a DUXB11 cell line or a DG44 cell line.

3. The system for over-expressing a target protein of claim 1, wherein the antifolate analog is Methotrexate (MTX) or Methionine sulfoximine (MSX).

4. The system for over-expressing a target protein of claim 1, wherein the first promoter is CMV promoter or SV40 promoter.

5. The system for over-expressing a target protein of claim 1, wherein the second promoter is CMV promoter or SV40 promoter, and the second promoter and the first promoter are different.

6. The system for over-expressing a target protein of claim 1, wherein the third promoter is U6 promoter.

7. The system for over-expressing a target protein of claim 1, wherein the fourth promoter is CMV promoter or SV40 promoter.

8. The system for over-expressing a target protein of claim 1, wherein the dCas9 expression cassette further comprises a 2A peptide sequence for linking the dCas9-KRAB gene and the antibiotic resistance gene.

9. The system for over-expressing a target protein of claim 1, wherein the antibiotic resistance gene is Zeocin resistance (Zeo^{R}) gene.

10. A method for over-expressing a target protein, comprising:
constructing a target protein expression plasmid, which comprises:
a target protein expression cassette, which comprises a first promoter and a target protein gene; and
a DHFR expression cassette, which comprises a second promoter and a DHFR gene;
constructing a CRISPRi expression plasmid, which comprises:
a gRNA cassette, which comprises a third promoter, a gRNA sequence and a terminator; and
a dCas9 expression cassette, which comprises a fourth promoter, a dCas9-KRAB gene and an antibiotic resistance gene;
establishing a first stable cell line by transfecting the target protein expression plasmid into a DHFR-deficient CHO cell and then screening with a screen medium to obtain the first stable cell line;
establishing a second stable cell line by transfecting the CRISPRi expression plasmid into the first stable cell line and then screening with an antibiotic to obtain the second stable cell line; and
performing a gene amplification by culturing the second stable cell line in a medium containing an antifolate analog for over-expressing the target protein.

11. The method for over-expressing a target protein of claim 10, wherein the DHFR-deficient CHO cell is a DUXB11 cell line or a DG44 cell line.

12. The method for over-expressing a target protein of claim 10, wherein the screen medium is a nucleoside-free α-MEM.

13. The method for over-expressing a target protein of claim 10, wherein the antibiotic is Zeocin.

14. The method for over-expressing a target protein of claim 10, wherein the antifolate analog is Methotrexate (MTX) or Methionine sulfoximine (MSX).


SEQUENCE LISTING (19 sequences)

SEQ ID NO: 1 - DNA, 584 bp, Artificial Sequence
  Synthetic CMV promoter
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 2 - DNA, 561 bp, Cricetulus griseus
atggttcgaccattgaactgcatcgtcgccgtgtcccaaaatatggggattggcaagaacggagacctaccctggcctccgctcaggaacgagttcaagtacttccaaagaatgaccacaacctcttcagtggaaggtaaacagaatctggtgattatgggtaggaaaacctggttctccattcctgagaagaatcgacctttaaaggacagaattaatatagttctcagtagagaactcaaagaaccaccacgaggagctcattttcttgccaaaagtttggatgatgccttaagacttattgaacaaccggaattggcaagtaaagtagacatggtctggatagtcggaggcagttctgtttaccaggaagccatgaatcaaccaggccacctcagactctttgtgacaaggatcatgcaggaatttgaaagtgacacgtttttcccagaaattgatttggggaaatataaacttctcccagaatacccaggcgtcctctctgaggtccaggaggaaaaaggcatcaagtataagtttgaagtctacgagaagaaaggc

SEQ ID NO: 3 - DNA, 720 bp, Artificial Sequence
  Synthetic EGFP gene
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 4 - DNA, 66 bp, Artificial Sequence
  Synthetic P2A peptide
ggatctggcgccaccaacttcagcctgctgaagcaggcaggcgacgtggaagagaaccctggccct

SEQ ID NO: 5 - DNA, 233 bp, Artificial Sequence
  Synthetic U6 promoter
tttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacg

SEQ ID NO: 6 - DNA, 48 bp, Artificial Sequence
  Synthetic terminator
ttttttgttttagagctagaaatagcaagttaaaataaggctagtccg

SEQ ID NO: 7 - DNA, 4452 bp, Artificial Sequence
  Synthetic dCas9-KRAB gene
atggacaagaagtactccattgggctcgctatcggtaccaacagcgtcggctgggccgtcattacggacgagtacaaggtgccgagcaaaaaattcaaagttctgggcaataccgatcgccacagcataaagaagaacctcattggagccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagctggtagacagtactgataaggctgacttgcggttgatctatctcgcgctggcgcacatgatcaaatttcggggacacttcctcatcgagggggacctgaacccagacaacagcgatgtcgacaaactctttatccaactggttcagacttacaatcagcttttcgaggagaacccgatcaacgcatccggcgttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagctgcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagctgaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgctcggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagagacgccgatatacaggatgggggcggctgtcaagaaaactgatcaatggcatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaaccggttgtccgactacgacgtggatgctatcgtgccccaaagctttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagctggaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccgcaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatccagcttcgagaaaaaccccatcgactttctcgaagcgaaaggatataaagaggtcaaaaaagacctcatcattaagctgcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgagttctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacggcaccggcgggcccaagaagaagaggaaggtatacccatacgatgttcctgactatgcgggctatccctatgacgtcccggactatgcaggatcgtatccttatgacgttccagattacgctatggacgcgaaatcacttacggcatggtcgagaacactggttacgttcaaggacgtgtttgtggactttacacgtgaggagtggaaattgctggatactgcgcaacaaattgtgtatcgaaatgtcatgcttgagaattacaagaacctcgtcagtctcggataccagttgacgaaaccggatgtgatccttaggctcgaaaagggggaagaaccttggctggta

SEQ ID NO: 8 - DNA, 372 bp, Artificial Sequence
  Synthetic Zeocin resistance (ZeoR) gene
atggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggac

SEQ ID NO: 9 - DNA, 225 bp, Artificial Sequence
  Synthetic BGH polyA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 10 - DNA, 20 bp, Artificial Sequence
  Synthetic gRNA of pCRISPRi-?
cgggtcttcgagaagacctg

SEQ ID NO: 11 - DNA, 20 bp, Artificial Sequence
  Synthetic gRNA of pCRISPRi-T
gcaagaacggagacctaccc

SEQ ID NO: 12 - DNA, 20 bp, Artificial Sequence
  Synthetic gRNA of pCRISPRi-NT
acggcgacgatgcagttcaa

SEQ ID NO: 13 - DNA, 20 bp, Artificial Sequence
  Synthetic Q mDHFR F
gaatcaaccaggccacctca

SEQ ID NO: 14 - DNA, 22 bp, Artificial Sequence
  Synthetic Q mDHFR R
acttgatgcctttttcctcctg

SEQ ID NO: 15 - DNA, 358 bp, Artificial Sequence
  Synthetic SV40 promoter
ctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaa

SEQ ID NO: 16 - DNA, 20 bp, Artificial Sequence
  Synthetic Q dCas9 F
aatggcatccgagacaagca

SEQ ID NO: 17 - DNA, 20 bp, Artificial Sequence
  Synthetic Q dCas9 R
tgtgctcgtgaagactgtcc

SEQ ID NO: 18 - DNA, 21 bp, Artificial Sequence
  Synthetic Q EGFP F
tgaacttcaagatccgccaca

SEQ ID NO: 19 - DNA, 20 bp, Artificial Sequence
  Synthetic Q EGFP R
ttctcgttggggtctttgct